SEQUENCE LISTING (9 sequences)

SEQ ID NO: 1 - protein, 17 aa, artificial sequence
  Synthetic
YTLNFRFWSDHISWYSC

SEQ ID NO: 2 - protein, 13 aa, artificial sequence
  Synthetic
YAISYRGKTTLLC

SEQ ID NO: 3 - protein, 14 aa, artificial sequence
  Synthetic
YHQWSYRGATYRSC

SEQ ID NO: 4 - protein, 16 aa, artificial sequence
  Synthetic
YNGPSYYRLRSWLSSC

SEQ ID NO: 5 - protein, 15 aa, artificial sequence
  Synthetic
YWITHSYRGLSKSFC

SEQ ID NO: 6 - protein, 14 aa, artificial sequence
  Synthetic
YQTWSWRGRRYSFC

SEQ ID NO: 7 - protein, 17 aa, artificial sequence
  Synthetic
YLHDSAFSFGWSLRLYC

SEQ ID NO: 8 - protein, 14 aa, artificial sequence
  Synthetic
YRQWSFRGRTFWSC

SEQ ID NO: 9 - protein, 15 aa, artificial sequence
  Synthetic
GLNDIFEAQKIEWHE

(19) United States
(12) Patent Application Publication
(10) Pub. No.: US 2024/0228543 A1
(43) Pub. Date: Jul. 11, 2024

(54) CYCLIC PEPTIDE INHIBITORS OF USP22

(71) Applicant: THE JOHNS HOPKINS UNIVERSITY, Baltimore, MD (US)

(72) Inventors: Cynthia Wolberger, Baltimore, MD (US); Michael T. Morgan, Baltimore, MD (US); Hiroaki Suga, Baltimore, MD (US)

(21) Appl. No.: 18/557,801

(22) PCT Filed: Apr. 29, 2022
(86) PCT No.: PCT/US22/27017

§ 371 Date: Oct. 27, 2023

Related U.S. Application Data
(60) Provisional application No. 63/276,353, filed on Nov. 5, 2021.
(60) Provisional application No. 63/182,148, filed on Apr. 30, 2021.

(51) Int. Cl. C07K 7/56; A61K 38/00

(52) U.S. Cl. CPC C07K 7/56; A61K 38/00

(57) ABSTRACT

The present disclosure provides peptide inhibitors of Ubiquitin-Specific Protease 22 (USP22), compositions, and methods of use thereof.

### CROSS-REFERENCE TO RELATED APPLICATIONS

This application claims the benefit of U.S. Provisional Application Nos. 63/182,148, filed Apr. 30, 2021, and 63/276,353, filed Nov. 5, 2021, the contents of which are herein incorporated by reference in their entirety.

### STATEMENT REGARDING FEDERALLY-SPONSORED RESEARCH OR DEVELOPMENT

This invention was made with government support under grant GM130393 awarded by the National Institutes of Health. The government has certain rights in the invention.

### FIELD

The present disclosure provides peptide inhibitors of Ubiquitin-Specific Protease 22 (USP22) and methods of use thereof.

### SEQUENCE LISTING STATEMENT

The text of the computer readable sequence listing filed herewith, titled “39503-601_SEQUENCE-LISTING_ST25”, created Apr. 29, 2022, having a file size of 2,374 bytes, is hereby incorporated by reference in its entirety.

### BACKGROUND

Histone ubiquitination serves a non-degradative role in regulating transcription. DNA repair. DNA replication, and chromatin condensation. Monoubiquitination of histone H2B (H2B-Ub) at lysine 120 in humans and lysine 123 in yeast is a hallmark of actively transcribed genes. H2B-Ub plays a variety of roles in transcription, including promoting assembly of the pre-initiation complex (PIC) and stimulating nucleosome reassembly by the histone chaperone. FAcilitates Chromatin Transcription (FACT). H2B-Ub also stimulates H3K4 methylation by human mixed lineage leukemia protein-1 (MLL1) and yeast COMPASS, and H3K79 methylation by human Dot1L and yeast Dot1. H2B-Ub is distributed throughout gene bodies and is particularly enriched at transcription start sites (TSSs). An intriguing aspect of H2B-Ub is that it is a transient mark that is rapidly turned over during transcription, with both ubiquitination and deubiquitination of H2B important for maintaining wild type levels of transcription. Deubiquitination of histone H2B plays a role in promoting phosphorylation of the RNA polymerase II CTD by Ctk1, thereby promoting the elongation phase of transcription. Studies have shown that genes with low H2B-Ub levels are expressed at higher levels than those with high H2B-Ub.

Altered levels of the enzymes that both attach and remove H2B-Ub are implicated in oncogenesis. Ubiquitin is covalently linked to H2B-K120 in human cells by the E2 ubiquitin conjugating enzyme, hRAD6A/B and the E3 ubiquitin ligase, RNF20/40. Misregulation of both the E2 and E3 enzymes that monoubiquitinate H2B have been shown to result in overexpression of genes that drive cell proliferation. A number of different deubiquitinating enzymes (DUBs) remove ubiquitin from H2B at distinct subsets of genes, including USP22, USP51, USP27x, USP36, and USP44. Of these DUBs, altered expression of USP22 has been implicated in a variety of tumor types. USP22 overexpression has been identified as part of an 11-gene “death by cancer” signature of gene expression—a pattern observed over diverse tissue types that is characteristic of metastatic cancers that respond poorly to existing therapies. USP22 overexpression correlates with poor clinical outcomes in tumors of the brain, breast, stomach, liver, and colon, although its mechanistic role in oncogenesis is poorly understood. In addition, reduced expression of USP22 is associated with chromosomal instability (CIN) because efficient chromosome compaction during mitosis requires USP22 deubiquitination of H2B in metaphase. Silencing of USP22 expression results in aberrant chromosomal segregation and phenotypes consistent with CIN, including polyploid daughter cells. Thus, USP22 activity is a regulator of major cellular events, and its misregulation is correlated with oncogenesis and genomic heterogeneity.

### SUMMARY

Disclosed herein are cyclic peptides, or pharmaceutically acceptable salts or solvates thereof, comprising an amino acid sequence with at least 50% similarity to any of SEQ ID NOs: 1-6. Also disclosed are compositions comprising the cyclic peptides disclosed herein. In some embodiments, the N-terminal amino acid is covalently linked to the C-terminal amino acid. In some embodiments, the covalent linkage is head to tail between the free C-terminal carboxyl and the free N-terminal amine. In some embodiments, the covalent linkage involves a side chain of the N-terminal amino acid, a side chain of the C-terminal amino acid, or both. In some embodiments, the N-terminal amino acid is covalently linked to the C-terminal amino acid with a thioether bond.

In some embodiments, the peptides are selected from

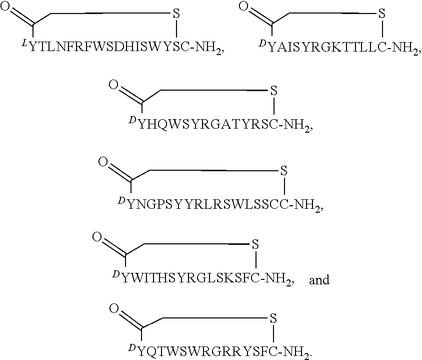

Further disclosed are methods of treating a disease or disorder comprising administering to a subject in need thereof an effective amount of a cyclic peptide or composition disclosed herein. In some embodiments, the disease or disorder comprises cancer.

Further disclosed are methods for modulating histone H2B ubiquitination comprising contacting a deubiquitinating enzyme with an effective amount of a cyclic peptide or composition disclosed herein. In some embodiments, the deubiquitinating enzyme comprises Ubiquitin-Specific Protease 22 (USP22). In some embodiments, the deubiquitinating enzyme comprises a member of a deubiquitinating protein complex. In some embodiments, the deubiquitinating protein complex is SAGA (Spt-Ada-Gen5-acetyltransferase) deubiquitinating module (DUBm).

Other aspects and embodiments of the disclosure will be apparent in light of the following detailed description and accompanying figures.

### BRIEF DESCRIPTION OF THE DRAWINGS

FIGS. 1A-1B show the random nonstandard Peptides Integration Discovery (RaPID) cyclic peptide library screening. FIG. 1A is a schematic of RaPID platform used for targeting the SAGA DUB module for novel binding species. FIG. 1B shows sequences for each peptide selected from RaPID screen for further evaluation as inhibitors. Each peptide contains the indicated D- or L-amino acid at the N-terminus covalently linked to the C-terminal cysteine sulfhydryl group.

FIGS. 2A-2G show inhibition of human DUB module activity by cyclic peptides. FIG. 2A is a graph of initial rates of Ub-AMC cleavage by hDUB module as a function of substrate concentration. Values of k_{cat} and K_{M} are indicated. FIGS. 2B-2G are graphs of each cyclic peptide, as indicated, tested for its ability to inhibit Ub-AMC cleavage in the presence of increasing amounts of the peptide into a reaction with a fixed amount of DUB (200 nM) and Ub-AMC (1 μM). Kᵢ values were determined for each curve using the Morrison Ki approximation.

FIGS. 3A-3B show inhibition of human DUB module activity on H2B ubiquitinated nucleosomes. FIG. 3A is images of Western blots showing the effect of each cyclic peptide (1 uM) and DMSO control on hDUB module on nucleosomes. FIG. 3B is a graph of the quantitation of the disappearance of the H2B-Ub band, performed in triplicate.

FIGS. 4A-4C show small angle x-ray scattering (SAXS) analysis of the DUB module in the presence and absence of cyclic peptide. FIG. 4A is a graph of scattering intensities of 1 mg/mL (6 μM) hDUBm alone (black), hDUBm in the presence of 7 uM hD1 (red), and theoretical scattering intensities calculated from the yDUBm crystal structure (PDB ID: 3MHH, cyan). The right y-axis describes raw measured intensities (upper curve), while the left y-axis is a plot of I*s values (Kratky plot). FIG. 4B is a plot of frequency distribution (P(r)) of possible vectors between surfaces on the scattering sample. Experimental curves from hDUBm (black) and hDUBm plus hD1 inhibitor (red); calculated curve for yDUBm (cyan). Dₘₐₓ is indicated by a colored arrow corresponding to each sample. FIG. 4C is electron density maps derived from SAXS data of hDUBm alone (gray) and hDUBm in the presence of hD1 (red). Dashed line indicates Dₘₐₓ. Correlations between the peptide-bound and apo complexes indicated by pseudocoloring (scale below). The yeast DUB module structure (green) was used to independently orient both density maps.

FIG. 5 is a bar graph showing end-point assays of indicated DUB in the presence or absence of inhibitor, hD1. For each, 1 μM Ubiquitin AMC was cleaved by either 200 nM hDUBm, 100 nM USP7CD, 100 nM CYLD, or 200 nM USP30 in the presence of either DMSO (mock control) or hD1.

FIGS. 6A-6B show that cyclic peptide inhibitors of hDUBm increase cellular H2B-Ub levels. FIG. 6A shows histone proteins extracted from HEK293T cells incubated with 5 μM of each cyclic peptide and analyzed by western blot with anti-H2Bub antibody. Bottom: anti-histone H3 loading control. FIG. 6B is a graph of the quantitation of western results indicates significant enrichment of H2B-Ub when normalized to H3 abundance. Bars labeled with two asterisks indicate p<0.01 (hD1 and hD5), and a single asterisk indicates p<0.05 (hD2, hD3, hD4, and PR-619) when each measurement is compared with untreated cells.

FIGS. 7A-7D are graphs of the selection of hDUBm from ^{L}Y-library (FIG. 7A) and ^{D}Ylibrary (FIG. 7B), and for yDUBm from ^{L}Y-library (FIG. 7C) and ^{D}Y-library (FIG. 7C). Red (right hand side) and blue (left hand side) bars represent the recovery rate of the cDNAs eluted from peptide-mRNA complex binding to DUBm-immobilized magnetic beads and biotin bound magnetic beads, respectively.

FIGS. 8A and 8B shows that yD1 does not inhibit the human or yeast DUB modules. In FIG. 8A, as in FIG. 2, hDUBm was subjected to a test of its enzymatic activity in the presence of a range of yD1 concentrations, yD1 was selected in RaPID screening against the yeast DUB module, and thus a negative result was expected, yD1 was further used as a negative control in which a cyclic peptide is present but inactive. FIG. 8B is a graph of 50 nM yeast DUB module incubated with either DMSO (vehicle) or 2.5 μM yD1 the peptide with the tightest binding to the yeast DUB module in RaPID screening. Each enzyme mixture was then mixed with a range of Ub-AMC concentrations, and cleavage was monitored by fluorescence. Rather than inhibition, yD1 produced a slight increase in cleavage rates.

FIGS. 9A-9C show that human DUB complex produced pairwise vector distributions [p(r)] calculated from SAXS data collected in saturating amounts of each indicated peptide compared with data collected without peptide (checked fill). Cyan portions of the curve fill indicate differences in the p(r) distribution due to peptide binding. In the case of hD1, peptide binding apparently contracts the complex at regions indicated by the grey checkered regions.

FIG. 10 is uncropped Western blot images of anti-H2B-Ub and anti-H3 blots of identical gels normalized to H3 signals. HEK293T cells were incubated with 5 μM of each indicated peptide. Each image contains two blots from two gels imaged side-by-side with three biological replicates of the indicated treatment. Lanes cropped as representative images for FIG. 6 are indicated with boxed overlays.

FIGS. 11A and 11B show that hD1 is a non-competitive inhibitor requiring the cyclized form for optimal USP22 targeting. FIG. 11A is a graph of hDUBm cleavage rates were measured at multiple substrate and hD1 concentrations to determine inhibitor equilibrium binding constant (Ki) and mode of inhibition. The data were best fit to a non-competitive model of enzyme inhibition, with a measured Km of 35 μM, kcat of 2.8 s⁻¹, and Ki of 180 nM. FIG. 11B is a graph of cleavage rates of 5 μM Ub-AMC by 200 nM hDUBm in the presence of the indicated concentration of either linear, scrambled, or cyclic hD1 peptide sequences fitted using IC₅₀ analysis. IC₅₀ values were calculated as 270 nM for the cyclic hD1, 1 μM for the linear version, and >1000 μM for the scrambled hD1 sequence.

FIGS. 12A and 12B show that hD1 is specific for USP22. FIG. 12A is a graph of the DUBProfiler assay of the effect of hD1 on a panel of 44 human DUBs. Cleavage of a ubiquitin-rhodamine(110)-glycine substrate was compared in the presence and absence of 1 μM hD1. FIG. 12B is a graph of the inhibition of H2B DUB complexes as a function of hD1 concentration. Cleavage rates of 5 μM Ub-AMC were assayed for 75 nM USP22/ATXN7L3/ENY2/ATXN7 (hDUBm), 100 nM USP27x/ATXN7L3/ENY2, or 1000 nM USP51/ATXN7L3/ENY2. Rates were normalized to the uninhibited reaction in each case to determine the fraction of apparent activity. IC₅₀ values of hD1 were 100 nM for USP22, 3 μM for USP27x, and >250 μM for USP51.

### DETAILED DESCRIPTION

The present disclosure provides cyclic peptides that are potent and highly specific inhibitors of deubiquitinating enzymes, e.g., USP22. Peptide binding did not impact the overall integrity of the deubiquitinating module (DUBm) complex as judged by small-angle x-ray scattering, indicating that the inhibitors do not disrupt subunit interactions required for activity. Cells treated with the cyclic peptides described herein had increased levels of H2B monoubiquitination, demonstrating the ability of the cyclic peptides to enter human cells and inhibit H2B deubiquitination. Thus, the DUBm can be targeted for inhibition with high specificity by each cyclic peptide identified and the cyclic peptides show favorable drug-like properties.

Section headings as used in this section and the entire disclosure herein are merely for organizational purposes and are not intended to be limiting.

### 1. DEFINITIONS

The terms “comprise(s).” “include(s).” “having,” “has.” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

A “chemotherapeutic agent.” as used herein, refers to a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy. Examples of chemotherapeutic agents include, but are not limited to: cyclophosphamide, methotrexate, 5-fluorouracil, doxorubicin, docetaxel, daunorubicin, bleomycin, vinblastine, dacarbazine, cisplatin, carboplatin, paclitaxel, raloxifene hydrochloride, tamoxifen citrate, abemacicilib, everolimus, alpelisib, anastrozole, pamidronate, anastrozole, exemestane, capecitabine, epirubicin hydrochloride, eribulin mesylate, erlotinib, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, trastuzumab, temozolomide, rapamycin, and tucatinib.

The term “contacting” as used herein refers to bring or put in contact, to be in or come into contact. The term “contact” as used herein refers to a state or condition of touching or of immediate or local proximity. Contacting to a target destination, such as, but not limited to, an organ, tissue, cell, or tumor, may occur by any means of administration known to the skilled artisan.

A “peptide” or “polypeptide” is a linked sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide”, “oligopeptide.” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The recitations “sequence identity,” “percent identity.” “percent homology,” “percent similarity,” or, for example, comprising a “sequence 50% identical to” or “sequence with at least 50% similarity to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro. Ser, Thr, Gly, Val. Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (e.g., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990. J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

The term “amino acid” or “any amino acid” as used here refers to any and all amino acids, including naturally occurring amino acids (e.g., α-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include $3-amino acids (3 and 02), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) or homoSerine(Cl) in its unbonded form may take the form of 2-aminobutyric acid (Abu) when participating in an intramolecular bond according to the present invention.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine. Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is retained by the peptide.

In the case of less common or non-naturally occurring amino acids, unless they are referred to by their full name (e.g. sarcosine, ornithine, etc.), frequently employed three- or four-character codes are employed for residues thereof, including. Sar or Sarc (sarcosine, i.e. N-methylglycine). Aib (α-aminoisobutyric acid), Dab (2,4-diaminobutanoic acid), Dapa (2,3-diaminopropanoic acid), Y-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βPhe (β-homophenylalanine) and Bip (β,βdiphenylalanine), and Ida (Iminodiacetic acid).

The term “pharmaceutically acceptable salt” in the context of the present invention (pharmaceutically acceptable salt of a peptide described herein) refers to a salt which is not harmful to a patient or subject to which the salt in question is administered. It may suitably be a salt chosen. e.g., among acid addition salts and basic salts. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the peptides may also be quaternized with alkyl chlorides, bromides, and iodides such as methyl, ethyl, propyl, isopropyl, butyl, lauryl, myristyl, stearyl and the like. Other examples of pharmaceutically acceptable salts are described in “Remington's Pharmaceutical Sciences”, 17th edition, Alfonso R. Gennaro (Ed.), Mark Publishing Company. Easton, Pa., USA, 1985 (and more recent editions thereof), in the “Encyclopaedia of Pharmaceutical Technology”, 3^{rd} edition, James Swarbrick (Ed.), Informa Healthcare USA (Inc.). NY, USA, 2007, and in J. Pharm. Sci. 66: 2 (1977).

As used herein, the terms “providing”, “administering,” “introducing,” are used interchangeably herein and refer to the placement of the peptides or compositions of the disclosure into a subject by a method or route which results in at least partial localization to a desired site. The peptides or compositions can be administered by any appropriate route which results in delivery to a desired location in the subject.

The term “solvate” in the context of the present invention refers to a complex of defined stoichiometry formed between a solute (the peptide or pharmaceutically acceptable salt thereof described) and a solvent. The solvent in this connection may, for example, be water, ethanol, or another pharmaceutically acceptable, typically small-molecular organic species, such as, but not limited to, acetic acid or lactic acid. When the solvent in question is water, such a solvate is normally referred to as a hydrate.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein. “treat,” “treating” and the like means a slowing, stopping, or reversing of progression of a disease or disorder when provided a peptide or composition described herein to an appropriate subject. The term also includes a reversing of the progression of such a disease or disorder to a point of eliminating or greatly reducing the disease. As such, “treating” means an application or administration of the peptides or compositions described herein to a subject, where the subject has a disease or a symptom of a disease, where the purpose is to cure, heal, alleviate, relieve, alter, remedy, ameliorate, improve, or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. CYCLIC PEPTIDES

The present disclosure provides cyclic peptides, or a pharmaceutically acceptable salt or solvate thereof, comprising an amino acid sequence with at least 50% similarity (e.g., at least 55%, at least 60%, at least 65%, at least 70%, at least 75%, at least 80%, at least 85%, at least 90%, at least 95%, at least 98%, or at least 99% similarity) to any of SEQ ID NOs: 1-6. In some embodiments, the peptide comprises or consists of the amino acid sequence of any of SEQ ID NOs: 1-6. In some embodiments, the peptide comprises or consists of an amino acid sequence having 1, 2, 3, 4, 5, 6, 7 or 8 amino acids different than any of SEQ ID NOs: 1-6.

SEQ ID NO: 1
YTLNFRFWSDHISWYSC
SEQ ID NO: 2
YAISYRGKTTLLC
SEQ ID NO: 3
YHQWSYRGATYRSC
SEQ ID NO: 4
YNGPSYYRLRSWLSSC
SEQ ID NO: 5
YWITHSYRGLSKSFC
SEQ ID NO: 6
YQTWSWRGRRYSFC

In some embodiments, the peptide comprising an amino acid sequence with at least 50% similarity to any of SEQ ID NOs: 1-6 comprises conservative substitutions (e.g., one or more amino acids are replaced by another, biologically similar residue defined by polarity, charge, acidity, hydrophobicity, or chemical structure (e.g., aromaticity). In some embodiments, the peptide comprising an amino acid sequence with at least 50% similarity to any of SEQ ID NOs: 1-6 comprises radical substitutions (e.g., one or more amino acids are replaced by residue with different physiochemical properties defined by polarity, charge, acidity, hydrophobicity, or chemical structure (e.g., aromaticity).

The peptides can be cyclized by any method available to one of skill in the art. For example, the N-terminal and C-terminal ends can be condensed to form a peptide bond by known procedures. Functional groups present on the side chains of amino acids in the peptides can also be joined to cyclize the peptides of the invention. For example, functional groups that can form covalent bonds include —COOH and —OH; —COOH and —NH₂; and —COOH and —SH. Pairs of amino acids that can be used to cyclize a peptide include, Asp and Lys; Glu and Lys; Asp and Arg; Glu and Arg; Asp and Ser; Glu and Ser; Asp and Thr; Glu and Thr; Asp and Cys; and Glu and Cys. Other examples of amino acid residues that are capable of forming covalent linkages with one another include cysteine-like amino acids such Cys, hCys, β-methyl-Cys and Penicillamine (Pen), a non-proteinogenic an alpha-amino acid having the structure of valine substituted at the beta position with a sulfanyl group, which can form disulfide bridges with one another. Preferred cysteine-like amino acid residues include Cys and Pen. Other pairs of amino acids that can be used for cyclization of the peptide will be apparent to those skilled in the art.

The groups used to cyclize a peptide need not be amino acids. Examples of functional groups capable of forming a covalent linkage with the amino terminus of a peptide include carboxylic acids and esters. Examples of functional groups capable of forming a covalent linkage with the carboxyl terminus of a peptide include —OH, —SH, —NH₂ and —NHR where R is (C₁-C₆) alkyl, (C₁-C₆) alkenyl and (C₁-C₆) alkynyl.

In some embodiments, the N-terminal amino acid is covalently linked to the C-terminal amino acid in the cyclic peptide. In some embodiments, the covalent linkage is head to tail between the free C-terminal carboxyl and the free N-terminal amine. In some embodiments, the covalent linkage involves a side chain of the N-terminal amino acid, a side chain of the C-terminal amino acid, or both. In some embodiments, the N-terminal amino acid is covalently linked to the C-terminal amino acid with a thioether bond.

Preferably, the reaction conditions used to cyclize the peptides are sufficiently mild so as not to degrade or otherwise damage the peptide. Suitable groups for protecting the various functionalities as necessary are well known in the art (see, e.g., Greene & Wuts, 1991, 2nd ed., John Wiley & Sons, NY), as are various reaction schemes for preparing such protected molecules.

In a preferred conventional procedure, the cyclic peptides of the present invention may be synthesized by solid-phase synthesis and purified according to methods known in the art. Any of a number of well-known procedures utilizing a variety of resins and reagents may be used to prepare the cyclic peptides of the present invention.

In another embodiment, the present invention also contemplates peptides comprising D-amino acids. It is not intended that the present invention be limited to particular amino acids and particular D-isomers.

In some embodiments, the peptides are selected from

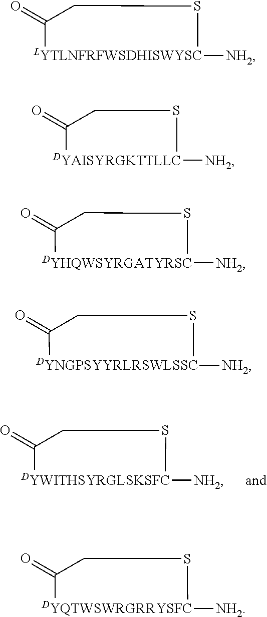

In some embodiments, the peptides are modified to stabilize them, to facilitate their uptake and/or absorption, or to improve any other characteristic or property of the peptides that is known to one of skill in art. For example, charges on the peptides can be neutralized and the peptides can be linked to other chemical moieties.

The peptides may also be modified by the addition of: radioactive atoms; detectable labels (e.g., radioactive labels, dyes, fluorescent moieties, chemiluminescent moieties, quantum dots); affinity tags (e.g., His tag, biotin); PEG moieties; carbohydrates (e.g., glycosylation, hesylation); and organic molecules (e.g., alkylation, acetylation, acylation).

The present disclosure also provides for nonpeptide compounds that mimic peptide sequences (“mimetics”), synthesis of which are known in the art. Peptide mimetics that are structurally related to therapeutically useful peptides may be used to produce an equivalent or enhanced therapeutic or prophylactic effect. Generally, peptidomimetics are structurally similar to the peptide of interest, but have one or more peptide linkages optionally replaced by linkages such as —CH₂NH—, —CH₂S—, —CH₂CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids. Peptides and Proteins, Weinstein eds. Marcel Dekker. New York). Such polypeptide mimetics may have significant advantages over naturally occurring polypeptides including more economical production, greater chemical stability, enhanced pharmacological properties (e.g., half-life, absorption, potency, efficiency), reduced antigenicity, and the like.

### 3. COMPOSITIONS

Disclosed herein are compositions comprising the peptides described above. The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any appropriate manner of administration, and thus administered, including for example, oral, nasal, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage. The route or administration and the form of the composition usually dictates the type of carrier to be used.

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

The disclosed peptides may be in a liposome in which peptide is combined with amphipathic agents such as lipids which exist in aggregated form as micelles, insoluble monolayers, liquid crystals, or lamellar layers which in aqueous solution. Suitable lipids for liposomal formulations include, without limitation, monoglycerides, diglycerides, sulfatides, lysolecithin, phospholipids, saponin, and bile acids. Preparation of such liposomal formulations is within the level of skill in the art.

### 4. METHODS

The disclosed peptides and compositions may be used in various methods, including methods for treating or preventing a disease or disorder in a subject or methods for modulating histone H2B ubiquitination.

In one embodiment, provided is a method for treating or preventing a disease or disorder in a subject comprising administering an effective amount of the peptides or compositions described herein to the subject. In some embodiments, the subject is a human.

The disclosed peptides of the invention are inhibitors of the SAGA (Spt-Ada-Gcn5-acetyltransferase) deubiquitinating module (DUBm). The SAGA DUBm comprises USP22 and three scaffolding proteins (ataxin 7 (ATXN7), ataxin-7-like protein 3 (ATXN7L3), and transcription and mRNA export factor ENY2). Thus, the disclosed peptides are useful in the treatment of diseases and disorders which are mediated or characterized by abnormal deubiquitination of histone H2B or overexpression of USP22. Characterized refers herein to a disease or disorder that results, entirely or partially, from abnormal deubiquitination of histone H2B or overexpression of USP22, or a disease or disorder wherein a particular symptom of the disease or disorder is caused, entirely or partially, by abnormal deubiquitination of histone H2B or overexpression of USP22.

In some embodiments, the disease or disorder is cancer. In some embodiments, the cancer comprises a solid tumor. In some embodiments, the cancer is metastatic cancer. In some embodiments, the disclosed peptides, compositions, or methods result in decreased tumor growth. In some embodiments, the disclosed peptides, compositions, or methods prevent tumor recurrence.

USP22 inhibitors may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. In some embodiments, the cancer may be brain cancer, breast cancer, stomach cancer, liver cancer, lung cancer, and colorectal cancer, or any combination thereof.

Peptides of the present disclosure may be administered to a subject by a variety of methods. In any of the uses or methods described herein, administration may be by various routes known to those skilled in the art, including without limitation oral, inhalation, intravenous, intramuscular, subcutaneous, systemic, and/or intraperitoneal administration to a subject in need thereof. In some embodiments, the peptides or compositions as disclosed herein may be administered by parenteral administration (including, but not limited to, subcutaneous, intramuscular, intravenous, intraperitoneal, intracardiac and intraarticular injections).

The terms “effective amount” or “therapeutically effective amount,” as used herein, refer to a sufficient amount of the peptides or compositions disclosed herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the disclosed peptides or compositions required to provide a clinically significant decrease in disease symptoms.

The amount of the peptides or compositions of the present disclosure required for use in treatment or prevention will vary not only with the particular peptide or composition selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of a peptide of the present invention, or composition thereof, can be determined by comparing their in vitro activity, and in vivo activity in animal models.

Dosage amount and interval may be adjusted individually to provide plasma levels of the active agent which are sufficient to maintain the modulating effects, or minimal effective concentration (MEC). The MEC will vary for each peptide but can be estimated from in vivo and/or in vitro data. Dosages necessary to achieve the MEC will depend on individual characteristics and route of administration. However, bioassays can be used to determine plasma concentrations. Dosage intervals can also be determined using MEC value. Compositions or peptides should be administered using a regimen, which maintains plasma levels above the MEC for 10-90% of the time, preferably between 30-90% and most preferably between 50-90%. In cases of local administration or selective uptake, the effective local concentration of the peptide may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be also used in veterinary medicine for non-human subjects.

Peptides and compositions disclosed herein can be evaluated for efficacy and toxicity using known methods. For example, the toxicology of a particular peptide may be established by determining in vitro toxicity towards a cell line, such as a mammalian, and preferably human, cell line. The results of such studies are often predictive of toxicity in animals, such as mammals, or more specifically, humans. Alternatively, the toxicity of particular peptides in an animal model, such as mice, rats, rabbits, dogs, or monkeys, may be determined using known methods. The efficacy of a particular peptide may be established using several recognized methods, such as in vitro methods, animal models, or human clinical trials. When selecting a model to determine efficacy, the skilled artisan can be guided by the state of the art to choose an appropriate model, dose, route of administration and/or regime.

A therapeutically effective amount of a peptide disclosed herein, or compositions thereof, may be administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time, wherein one composition includes a peptide of this invention, and the other includes the second agent(s). Alternatively, in other embodiments, the therapy precedes or follows the other agent treatment by intervals ranging from minutes to months.

A wide range of second therapies may be used in conjunction with the peptides of the present disclosure. The second therapy may be a combination of a second therapeutic agent or may be a second therapy not connected to administration of another agent. Such second therapies include, but are not limited to, surgery, immunotherapy (e.g., chimeric antigen receptor (CAR) T-cell or T-cell transfer therapies, cytokine therapy, immunomodulators, cancer vaccines, or administration of antibodies (e.g., monoclonal antibodies)), radiotherapy, or a chemotherapeutic agent.

The second therapy (e.g., an immunotherapy) may be administered at the same time as the initial therapy, either in the same composition or in a separate composition administered at substantially the same time as the first composition. In some embodiments, the second therapy may precede or follow the treatment of the first therapy by time intervals ranging from hours to months.

In another embodiment, provided is a method for modulating histone H2B ubiquitination comprising contacting a deubiquitinating enzyme with an effective amount of the peptides or compositions described herein. The modulation may result in an increase in histone H2B ubiquitination, or a decrease in H2B deubiquitination.

In some embodiments, the deubiquitinating enzyme is a member of the subfamily of ubiquitin-specific processing proteases (USPs). The deubiquitinating enzyme may comprise any deubiquitinating enzyme or related proteins which have catalytic activity for histone H2B, including, but not limited to USP22, USP51, USP27x, USP36, and USP44. In some embodiments, the deubiquitinating enzyme comprises USP22. In some embodiments, the deubiquitinating enzyme comprises a deubiquitinating protein complex. In some embodiments, the deubiquitinating protein complex is SAGA (Spt-Ada-Gcn5-acetyltransferase) deubiquitinating module (DUBm). In some embodiments, the deubiquitinating enzyme comprises USP22, ATXN7. ATXN7L3, and/or ENY2.

In some embodiments, the deubiquitinating enzyme is in a cell, such that contacting the deubiquitinating enzyme with the peptides or compositions described herein comprising introducing the peptides or compositions described herein into a cell comprising the targeted deubiquitinating enzyme. Methods for introducing into a cell are well known in the art and include, but are not limited to, chemical transfection, electroporation, microinjection, biolistic delivery via gene guns, or magnetic-assisted transfection.

In some embodiments, the cell is in an organism or host, such that introducing the peptides or compositions described herein into the cell comprises administration to a subject. The method may comprise providing or administering to the subject, in vivo, or by transplantation of cells treated ex vivo (e.g., in vitro) with the peptides or compositions as described elsewhere herein.

### 5. KITS

In another aspect, the disclosure provides kits comprising at least one disclosed peptide, or a composition comprising the peptide, and instructions for using the peptide or composition.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed peptide and/or product and another agent (e.g., a chemotherapeutic, a monoclonal antibody, a pain reliever, a steroid, an anti-emetic) for delivery to a patient or a cell.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates, flasks).

The kits provided herein are in suitable packaging. Suitable packaging includes, but is not limited to, vials, bottles, jars, flexible packaging, and the like. Individual member components of the kits may be physically packaged together or separately.

### 6. EXAMPLES

### Materials and Methods

Protein expression and purification For library screening, Ubp8 and USP22 constructs with N-terminal 6xHis were cloned into expression vectors, extending the N-terminus with an Avi-tag (GLNDIFEAQKIEWHE. SEQ ID NO: 9), an amino acid sequence that can be specifically biotinylated by the enzyme BirA during expression or in a reconstituted biochemical reaction. The yeast DUB module was expressed and purified as previously described (Samara. N. L., et al., (2010) Science 328, 1025-1029, incorporated herein by reference in its entirety). Briefly, bacterial cells (BL21-Rosetta2-pLysS) were transformed with three separate plasmids bearing the following coding sequences: Ubp8 (pET32a). Sgfl1 and Sgf73 1-105 (CDFDuet), and Susl (pRSF). Cells were harvested, resuspended in lysis buffer (50 mM HEPES pH 7.8, 500 mM NaCl, 20 mM imidazole, 50 μM ZnCl2, 5% glycerol, 15 mM betamercaptoethanol, and 1 mM PMSF) and lysed using a microfludizer (Microfluidics, Inc.) followed by passage over a HisTrap column (GE Healthcare) and elution with a 20-400 mM imidazole gradient in lysis buffer. Peak fractions were collected and dialyzed into IEX buffer (20 mM HEPES pH 7.6, 50 mM NaCl, 20 μM ZnCl₂, 15 mM betamercaptoethanol, and 5% glycerol), followed by further purification with a Q-SP column (GE Healthcare) using a 0.05-1 M salt gradient in the same buffer. Fractions containing the yeast DUB module were then pooled, concentrated, and injected onto a Superdex 200 10/300 column (GE Healthcare) equilibrated with storage buffer (20 mM HEPES pH 7.6, 150 mM NaCl, 20 μM ZnCl2, 5 mM DTT, and 5% glycerol). Peak fractions were pooled, concentrate/ed and flash-frozen in aliquots.

The human DUBm was expressed using a baculovirus plasmid construct containing all four subunits. USP22, ATXN7(3-151), ATXN7L3, and ENY2, using the Bac2Bac system (Thermo), which were used to transfect Sf9 insect cells in order to amplify the resultant baculovirus. High Five cells were infected with the hDUBm baculovirus for two days, harvested, lysed, and purified in the same buffers used for yDUBm. After purification, recombinant BirA was added to the purified complexes in the presence of 50 mM biotin to produce the biotinylated complex, which was suitable for immobilization and mRNA display screening.

All biochemical and biophysical assays used yDUBm or hDUBm constructs lacking the Avi tag, which were purified essentially as described for their Avi-tagged counterparts, with the exception that the 6xHis affinity tag was cleaved from Ubp8 and USP22 by addition of TEV protease following HisTrap purification and repassage of the ensuing product over the HisTrap column to remove the cleaved tag and protease.

Screening of DUBm binding macrocyclic peptides with the RaPID system In vitro selections of DUBm binding macrocyclic peptides using RaPID system was performed as previously reported (Ito. K., et al., Nat Commun 6, 6373, incorporated herein by reference in its entirety) with slight modification. Briefly, the initial random mRNA library was transcribed and ligated to a puromycin linker primer via T4 ligase for 30 min at 25° C. and extracted with phenol/chloroform and ethanol precipitated. A 150 μL translation reaction using the methionine-deficient FIT system and a 50 μM concentration of C1Ac-L-Tyr-tRNA^{fMet}_{CAU} and CIAc-D-Tyr-tRNA^{fMet}_{CAU} were used to convert the mRNA library into a library of peptide-mRNA fusions. The translation was performed at 37° C. for 30 min followed by a 25° C. step for 12 min to enhance the formation of the peptide-mRNA fusions. Thirty microliters of 100 mM EDTA was added to dissociate ribosomes and the peptide-mRNA fusions were incubated at 37° C. for 30 min to allow the thioether cyclizations to approach completion.

The fused peptide-mRNA was subsequently reverse transcribed using M-MLV Reverse Transcriptase RNase H-(Promega) for 1 h at 42° C. and 0.5 μL aliquot of the peptide-mRNA fusions was taken from the mixture and saved for the determination of the total amount of inputted mRNA. The peptide-mRNA fusions were then incubated with human and yeast DUBm-immobilized on Dynabeads M-280 streptavidin (Invitrogen) for 30 min at 37° C. The resultant complementary DNAs were eluted by mixing with 1×PCR reaction buffer and heating at 95° C. for 5 min. followed by immediate separation of the supernatant from the beads. A small fraction of the cDNA and input were allocated to real-time PCR quantification using a LightCycler 2.0 (Roche); the remainder was amplified by PCR. The resulting duplex DNAs were purified by phenol-chloroform extraction and ethanol precipitation, and transcribed into mRNAs for the next round of selection. From the second round of selection, the translation was performed at 5 μL scale, and six times of pre-clear steps were added as negative selection preceding the positive selection steps using 1 μL each of untreated and biotin bound Dynabeads. Finally, the observed enrichments appearing at fifth and sixth round were subjected to further DNA deep sequencing using the MiSeq sequencing system (Ilumina).

Chemical synthesis of peptides Macrocyclic peptides were synthesized by standard Fmoc solid-phase peptide synthesis (SPPS) using a Syro Wave automated peptide synthesizer (Biotage). The resulting peptide-resin (25 μmol scale) was treated with a solution of 92.5% trifluoroacetic acid (TFA), 2.5% water, 2.5% triisopropylsilane and 2.5% ethanedithiol, to yield the free linear N—C1Ac-peptide. Following diethyl ether precipitation, the pellet was dissolved in 10 ml triethylamine containing DMSO and incubated for 1 h at 25° C., to yield the corresponding macrocycle. The peptide suspensions were then acidified by addition of TFA to quench the macrocyclization reaction. The macrocycle was purified by RPHPLC, using a Prominence HPLC system (Shimadzu) under linear gradient conditions. Mobile phase A (comprising water with 0.1% TFA) was mixed with mobile phase B (0.1% TFA in acetonitrile). Purified peptides were lyophilized in vacuo and molecular mass was confirmed by MALDI MS, using an AutoFlex II instrument (Bruker Daltonics).

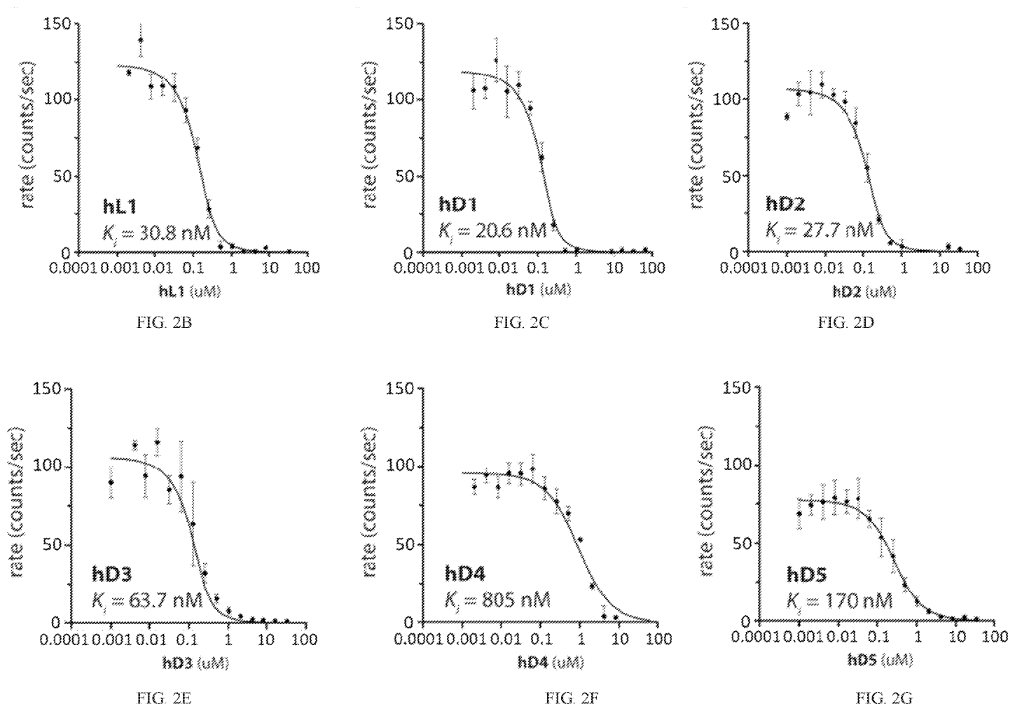

Ub-AMC deubiquitination assay A working stock mixture of either yDUBm or hDUBm was prepared in the presence of either DMSO (mock) or a dilution series of the cyclic peptide. Before making the stock mix, the enzyme complex was diluted in reaction buffer (50 mM HEPES, pH 7.5, 150 mM NaCl, 1 μM ZnCl2, and 5 mM DTI), and each cyclic peptide was serially diluted with DMSO from 1.6 mM in 2-fold increments to make 15× stocks of each desired final peptide concentration (so as to keep DMSO concentration constant in all reactions), then diluted in buffer before mixing with enzyme. Each enzyme:peptide mixture was incubated for 30 min at 30° C. in the case of yDUBm, and 37° C. in the case of hDUBm, with each enzyme complex at 1.033× the desired concentration (207 nM). The enzyme:peptide mixture (29 μM) was added to 1 μL of 30 μM U b-AMC (7-amino-4-methylcoumarin) (Boston Biochem), for a final concentration of 200 nM DUBm, the indicated cyclic peptide concentration, and 1 μM Ub-AMC. DUB activity was measured by observing the increase in AMC fluorescence in a plate reader, and the initial rate of fluorescence increase was calculated and plotted as a function of inhibitor concentration (FIGS. 2B-2I). The Ki values were determined using the Morrison approximation of enzyme inhibition equilibria within Prism (Graphpad).

Deubiquitination of nucleosomal H2B Recombinant human mononucleosomes containing histone H2B ubiquitinated at K120 (H2B-Ub) were obtained from Epicypher (dNuc, 16-0370) and diluted to 2 μM with reaction buffer. Both hDUBm and inhibitor peptides were diluted from concentrated stocks with reaction buffer (10 mM HEPES pH 7.5, 150 mM NaCl, 1 μM ZnCl2, and 0.2 mM TCEP) to a final concentration of 400 nM DUB module and 2 μM cyclic peptide and incubated at 37° C. for 20 minutes. Reactions were initiated by combining equal volumes of the enzyme-peptide mixture and nucleosomes, resulting in final concentrations of 200 nM hDUBm, 1 μM cyclic peptide, and 1 uM H2B-Ub nucleosome. Time points of the reaction were quenched with SDS sample buffer, separated by SDS-PAGE (Invitrogen, Bolt gels), and stained with Sypro Ruby (Life Technologies). Gels were imaged and quantitated (BioRad product info), using the H3 band as an independent load control to normalize the band intensities of H2B-Ub. Experiments were done in triplicate and analyzed using Prism (GraphPad).

Small-angle x-ray scattering Small angle x-ray scattering data (SAXS) were collected on a Rigaku BioSAXS 2000 instrument mounted on a Rigaku FRE-Super Bright rotating anode x-ray generator. The yeast and human DUBm and DUBm were diluted to concentrations of 1 mg/mL (6 μM) in reaction buffer (see H2B-Ub DUB assay) and scattering data was recorded with a Pilatus-6M detector. To record SAXS data from each DUBm in the presence of cyclic peptide, both peptide and DUBm were incubated at equimolar concentration (6 M). Data were analyzed using the Primus suite of SAXS analysis software to obtain p(r) curves. These data were then processed further using the DENSS algorithm to produce electron density maps. Maps were then further analyzed using UCSF Chimera.

Cell-based Assay HEK293T cells were purchased from ATCC (CRL-3216) and cultures were expanded for at least 10 passages in Dulbecco's Modified Eagle Medium (DMEM) supplemented with 10% fetal bovine serum (FBS) in T75 flasks incubated in a 37° C. and 5% CO₂ cabinet before any manipulation. Once cells were mature, HEK293T cells were grown to 80% confluence in 6-well plates, the media were aspirated, and media with reduced FBS (DMEM, 2% FBS) and 5 μM cyclic peptide were introduced. After two hours, the media were aspirated, and the cells were collected by scraping (˜1×10⁶ cells per well). The cells were then washed in 1× PBS twice followed by histone extraction (Active Motif). The extracted histones were then western blotted for H2B-Ub levels (Cell Signaling, 5546S), using H3 western blotting (Abcam, ab1791) to normalize loading.

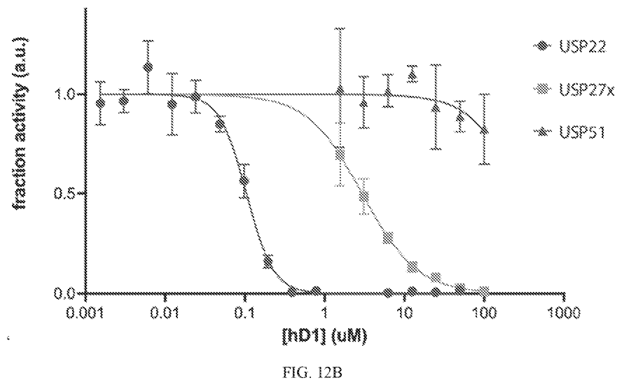

DUB specificity assays Ubiquitin-rhodamine(110)-glycine cleavage rates were measured for all DUBs available in the DUBprofiler™ panel with two independent replicates (44 in total; Ubiquigent). Enzyme activity was measured in the presence of a constant concentration of 1 μM hD1. USP22 was not included in the panel, and is not represented in FIG. 12A. Activity of purified DUB complexes targeting H2B that coopt ATXN7L3 and ENY2 was tested in the presence of a range of concentrations of hD1. Initial cleavage rates of each complex were measured on a fixed concentration of 5 μM Ub-AMC.

### Example 1

Screen for Macrocyclic Peptides that Inhibit the DUB Module

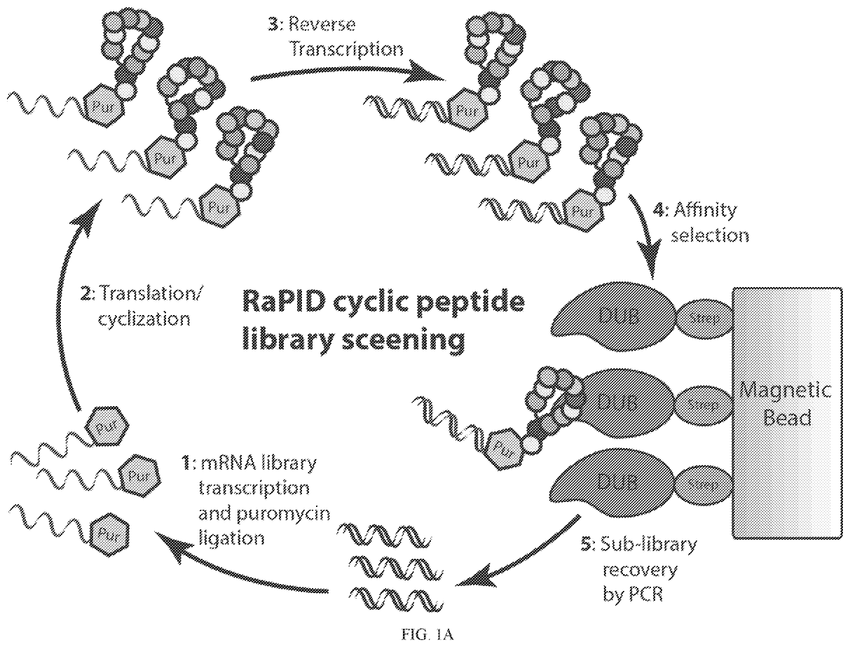

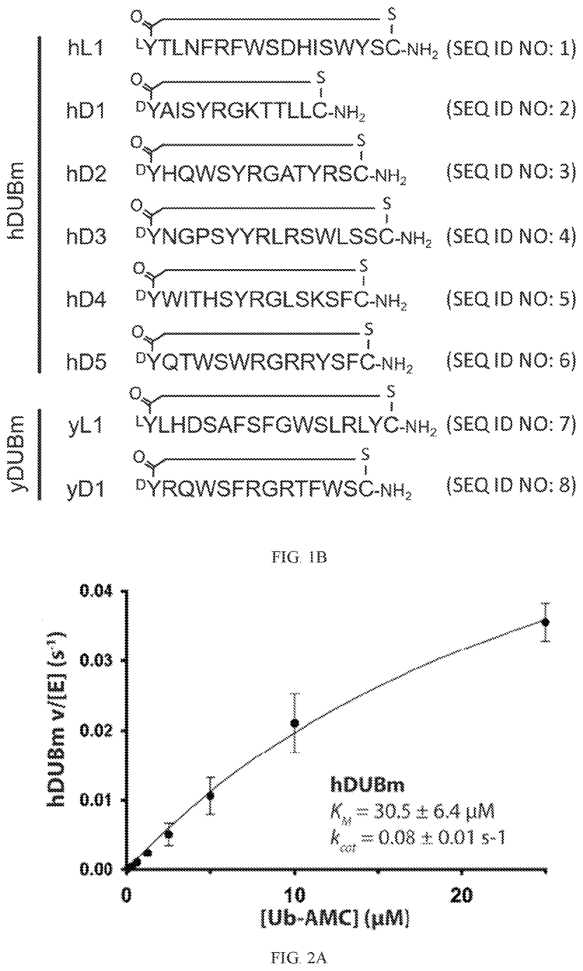

The RaPID system was employed to screen libraries of over 1012 unique macrocyclic peptides to identify candidate inhibitors based on their ability to bind tightly to the human DUB module (hDUBm) or yeast DUB modules (yDUBm) (FIG. 1A). A puromycin-ligated mRNA library was constructed to encode peptides with N-chloroacetyl-L-Tyrosine (^{L}Y-library) or N-chloroacetyl-D-Tyrosine (^{D}Y-library) as the initiator amino acid, followed by a random peptide region consisting of 6-15 residues, a cysteine and ending with a short linker peptide. Upon translation of these mRNAs, the chloroacetyl group on N-terminus of the linear peptides spontaneously cyclizes with the downstream cysteine to form thioether-macrocyclic peptides. The cyclic scaffold ensured that the ^{L}Y-library and the ^{D}Y-library diversified three-dimensional structures. Each cyclic peptide was covalently linked to its corresponding mRNA template via the puromycin linker for later amplification and DNA sequencing. These libraries were applied to streptavidin-conjugated magnetic beads to which a N-terminally biotinylated hDUBm or yDUBm was immobilized for selection of ligands against each enzyme complex.

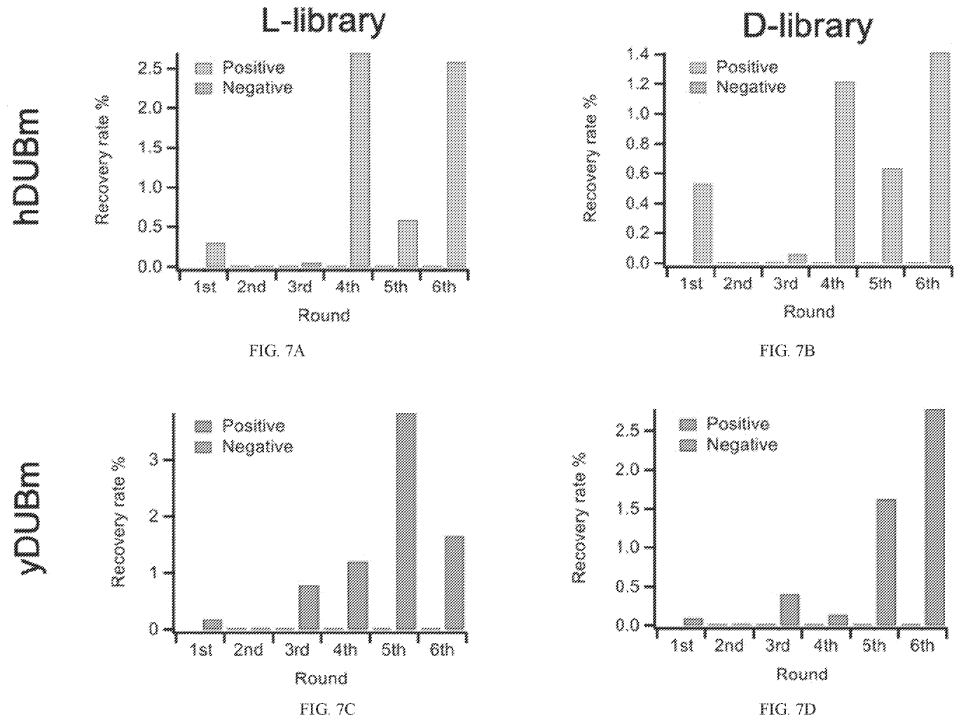

Following four or five rounds of selection, the recovery rate of peptide-mRNA fusion molecules was significantly increased (FIG. 7), suggesting that the population of cyclic peptides binding hDUBm or yDUBm were selectively enriched. Sequence analysis of the respective enriched libraries yielded unique sequences for the peptides. Six of the most enriched cyclic peptides for hDUBm and two sequences targeting yDUBm were selected on the basis of the hit frequency for further analysis.

### Example 2

### Peptides are Potent Inhibitors of the Human DUB Module

The cyclic peptides that constituted potential inhibitors were chemically synthesized by solid phase peptide synthesis and tested for their effectiveness and specificity as inhibitors in vitro. Since the enzyme kinetics of the human DUB module had not previously been characterized, hDUBm activity was assayed using the fluorogenic substrate, ubiquitin-amino methyl coumarin (Ub-AMC) (FIG. 2A). The ability of each cyclic peptide to inhibit DUB module cleavage of Ub-AMC cleavage was assayed over a range of concentrations under standardized enzymatic conditions. As shown in FIGS. 2B-2G, all six peptides that were identified based on their tight binding to the human DUB module were also potent inhibitors of DUB activity. All peptides had submicromolar Ki values, with the hL1, hD1, hD2, and hD3 peptides exhibiting Ki values of <100 nM. The most potent inhibitor, hD1, had an apparent Ki of 20.6 nM.

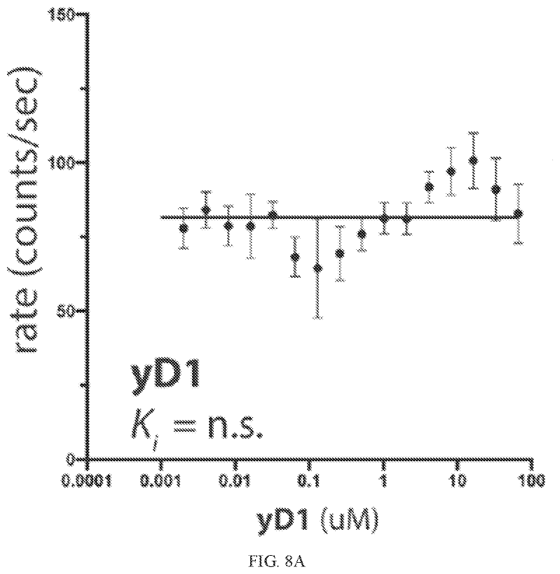

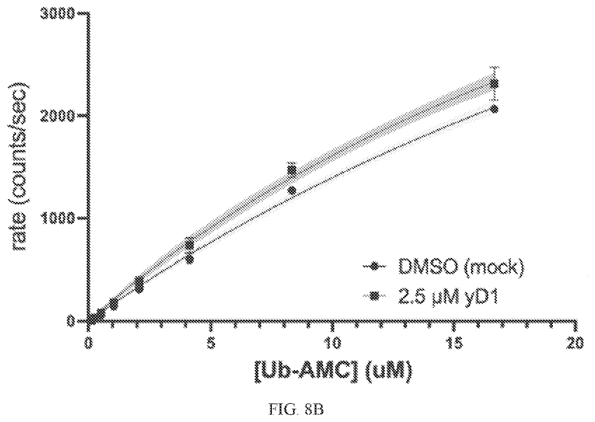

The two peptides targeting the yeast DUB module were not pursued as inhibitors, as yL1 was insoluble in aqueous conditions and yD1 did not inhibit activity of the yeast DUB module (FIG. 8A). Since yD1 also did not inhibit the human DUBm (FIG. 8B), nor was it enriched in the screen for peptides that bind tightly to the human DUBm, this peptide was used for control experiments.

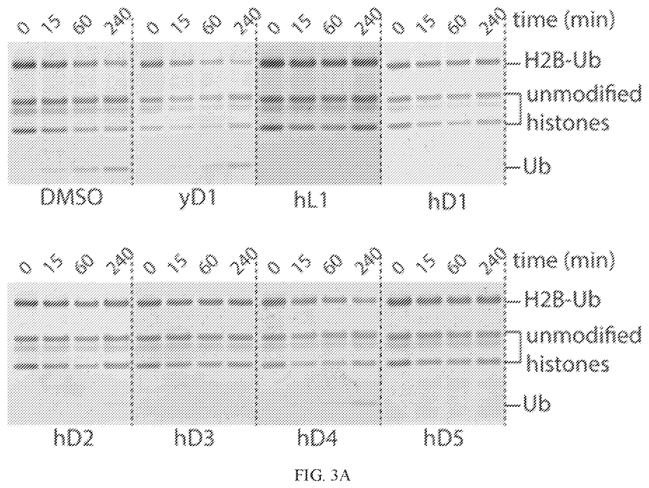

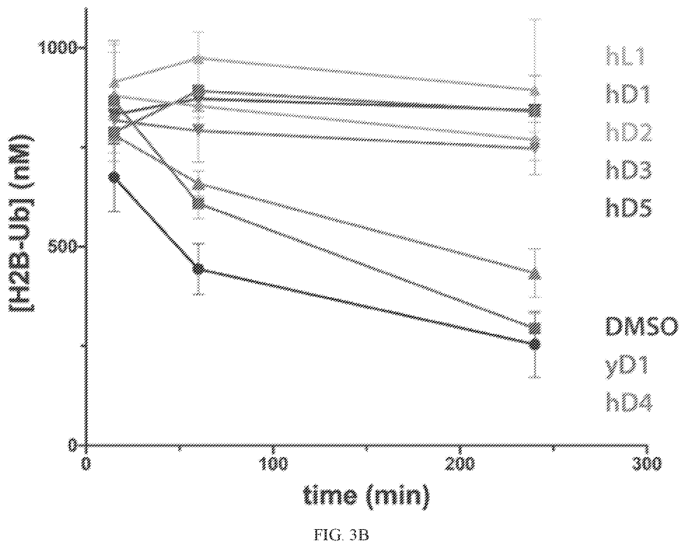

To test the effectiveness of the inhibitors on a native-like substrate, the ability of each cyclic peptide to inhibit DUB activity on nucleosomes ubiquitinated at H2B-K120 was assayed. A denaturing gel was used to monitor cleavage of H2B-Ub, releasing H2B and Ub (FIG. 3A). With the exception of hD4, all of the cyclic peptide leads were potent inhibitors of nucleosomal H2B-Ub cleavage by the human DUB module (FIGS. 3A and 3B). Each cyclic peptide sufficiently inhibited hDUB module activity such that no substantial activity was detected over the time course examined. The peptide with the highest Ki on Ub-AMC, hD4 (804 nM) (FIG. 2G), also showed very weak inhibition of hDUBm on nucleosomal H2B-Ub. These results confirmed the ability of cyclic peptides hL1, hD1, hD2, hD3, and hD5 to inhibit the enzymatic activity of USP22/DUB module on a native substrate.

### Example 3

### SAXS Studies of Human DUB Module Bound to Peptide Inhibitors

Most enzyme inhibitors disrupt activity by binding in or near the active site and interfering with substrate binding. Since USP22 is only active when bound to the other DUB module proteins, another possible mechanism by which the cyclic peptides inhibit USP22 activity could be by altering the interactions between USP22 and the three other subunits, ATXN7, ATXN7L3, and ENY2. Studies of the yeast DUB module showed that deletion of the Sgf11 zinc-finger leads to an inactive domain-swapped dimer of DUB modules, while mutations made at the interface between Ubp8 and the Sgf11 zinc finger significantly reduce enzyme activity. Similarly, disruption of inter-subunit interactions between the catalytic subunit, Ubp8 (yeast homologue of USP22), and Sgf73 (homologue of ATXN7) cause a loss of DUB activity. Small-angle x-ray scattering (SAXS) was used to determine whether the DUB module undergoes a large-scale change to its shape in the presence each respective cyclic peptides. Since there is no structure of the human DUB module, the results were compared to predicted SAXS data calculated from the crystal structure of the yeast DUB module (PDB ID: 3MHH).

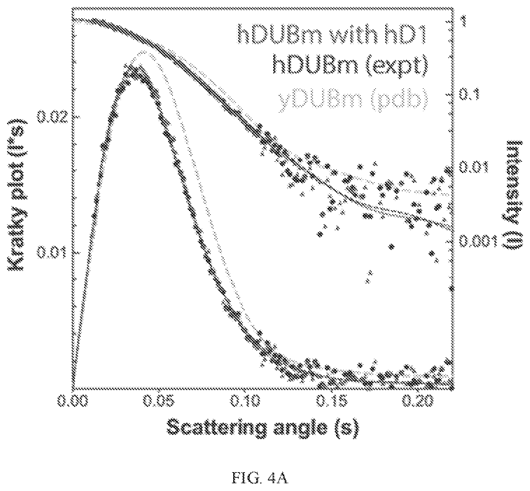

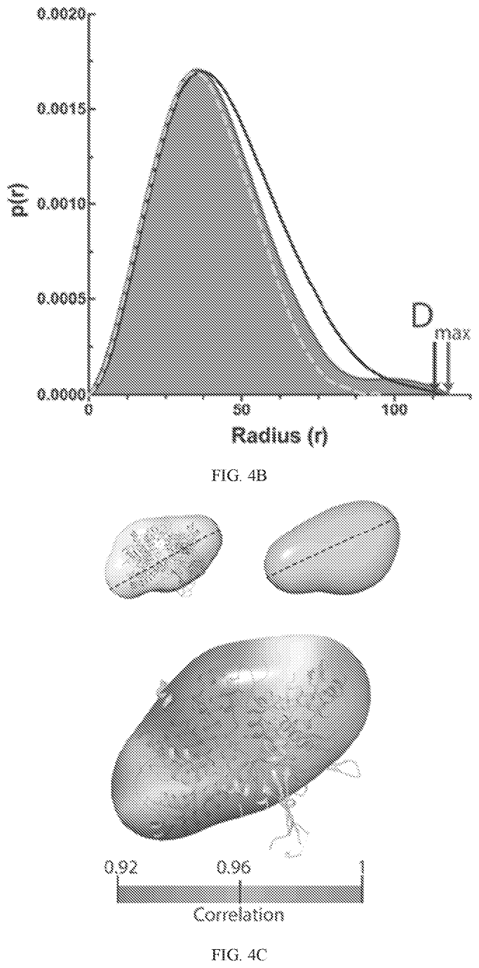

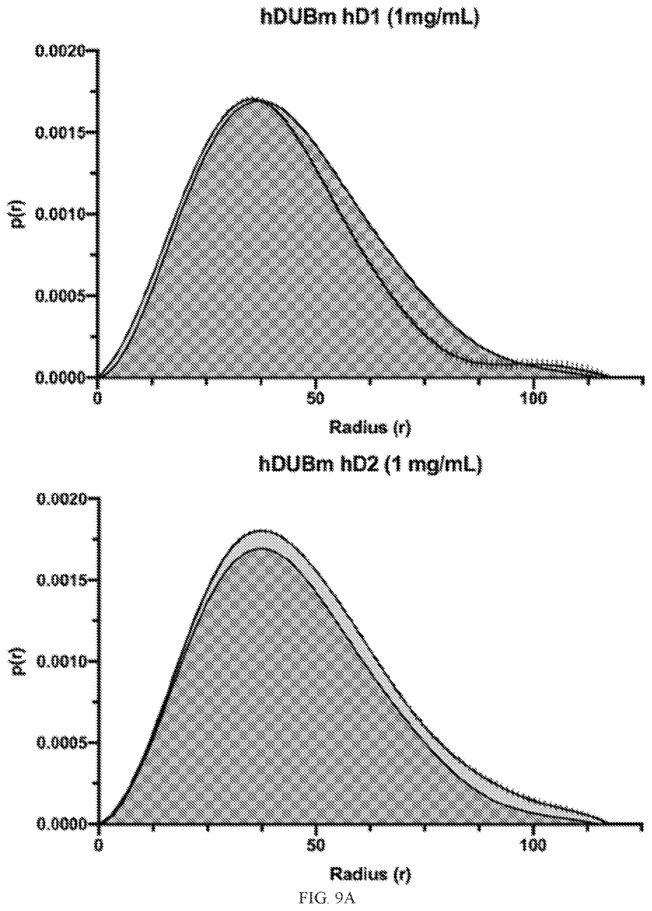

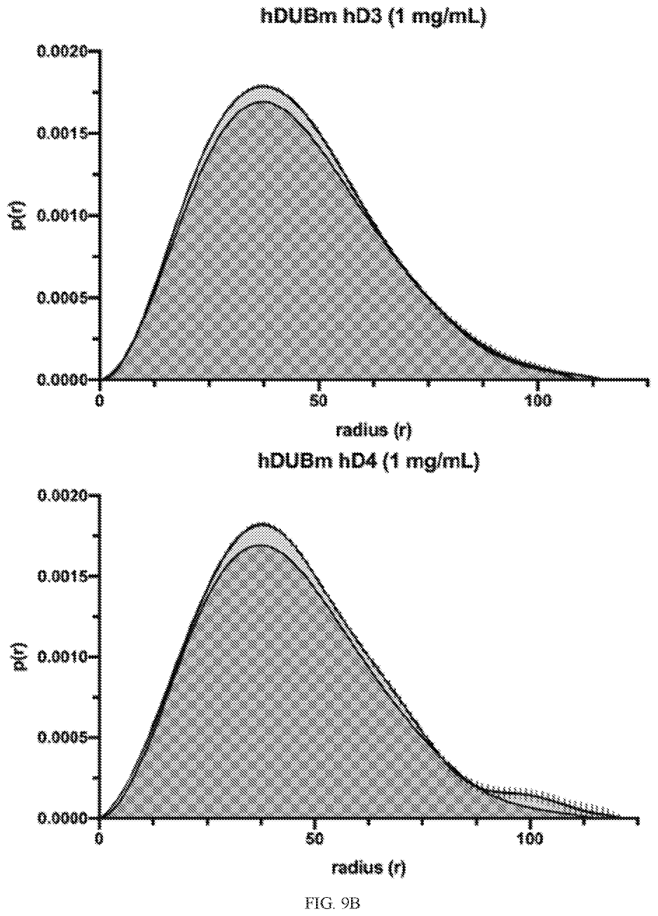

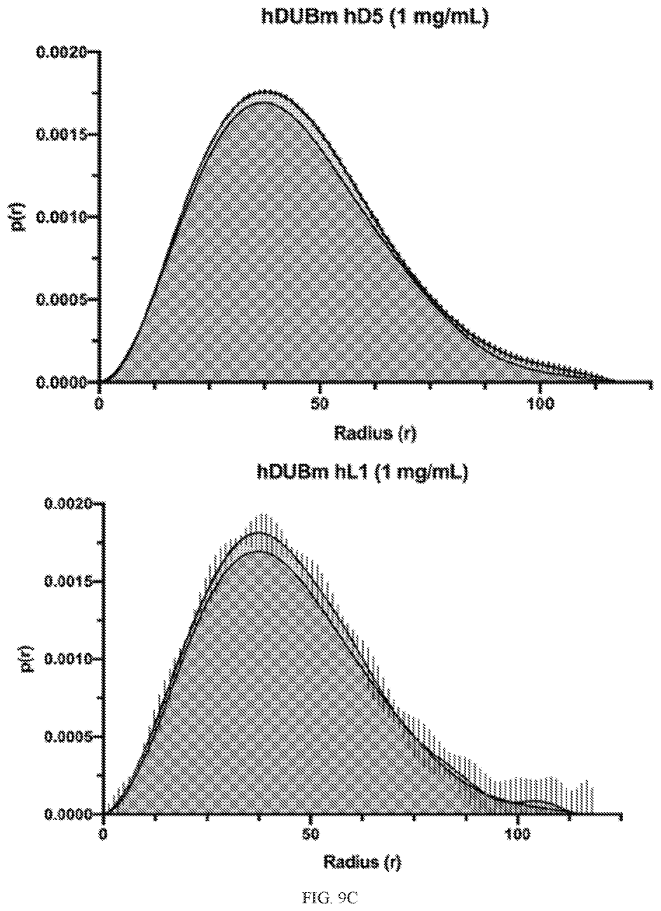

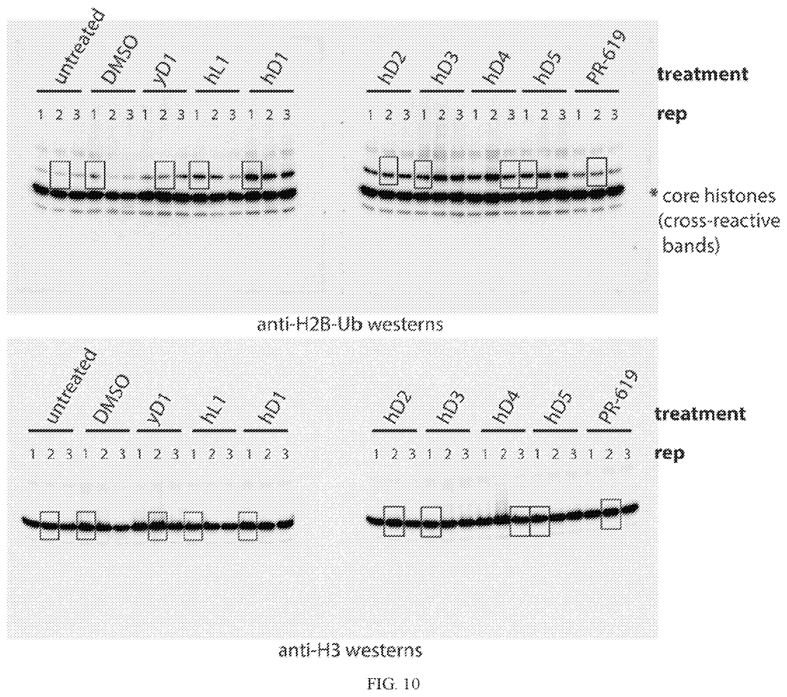

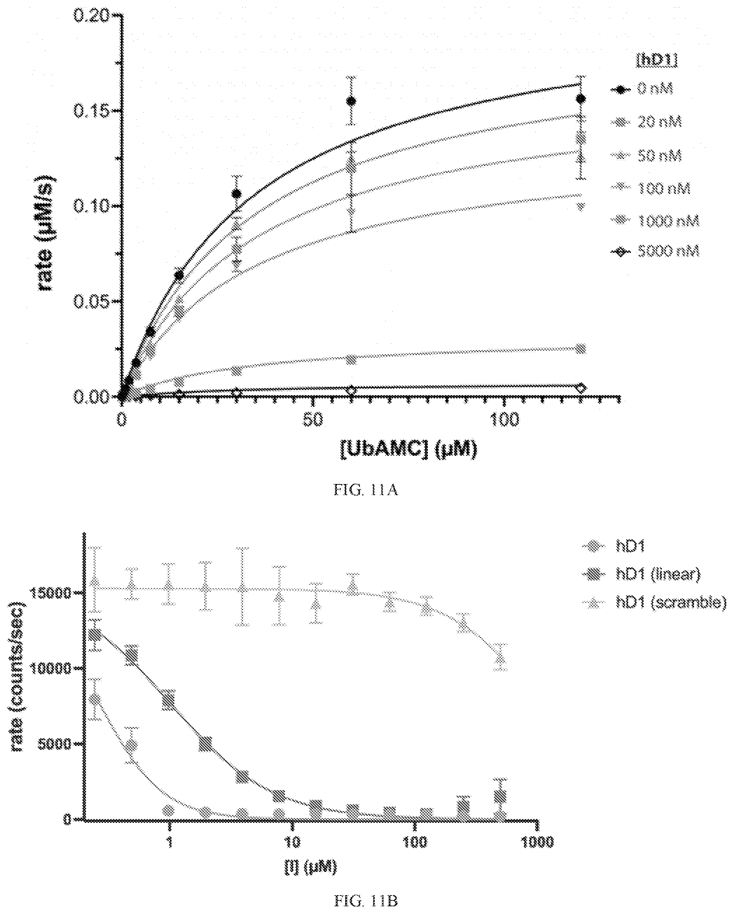

SAXS data were measured with the human DUB module incubated with each respective peptide inhibitor, using DMSO as a vehicle control. The Kratky plot (FIG. 4A) and p(r) distribution (FIG. 4B) of the human DUB module in the presence of the hD1 inhibitor were very similar to those of the yeast complex, consistent with the idea that the two complexes adopt a similar tertiary and quaternary structure. A similar trend was seen in SAXS data collected on the human DUB module in the presence of the other cyclic peptides (FIGS. 9A-9C), suggesting that the peptides appear to bind the DUB module at discrete surfaces, rather than disrupting the complex. Consistent with this conclusion, ab initio density maps calculated for the human and yeast DUB modules in the presence and absence of hD1 using DENSS analysis indicated minor changes in the molecular envelope. Interestingly, binding of each cyclic peptide slightly increased the long axis of the complex (FIG. 4C). The greatest difference in molecular envelope due to peptide binding occurred in the region of the Sgf11/ATXN7L3 zinc finger, which is located at one end of the long axis (FIG. 4C).

### Example 4

### Inhibitor is Selective for Human USP22

In order to probe the relative specificity of the USP22 inhibitors, the effect of hD1 was tested on three human DUBs: the USP7 catalytic domain (USP7CD), USP30, and CYLD. USP7 and USP30 are USP DUBs and belong to the same structural family as USP22, while CYLD belongs to the structurally distinct ubiquitin C-terminal hydrolase (UCH) family of DUBs. The activity of hDUBm and each off-target DUB was tested with DMSO (mock) or 1 μM hD1. Initial rates of ubiquitin-AMC cleavage were normalized for the intrinsic differences in each enzyme's activity. As shown in FIG. 5, the hD1 peptide had no effect on USP7CD or CYLD activity, and only a very modest (˜15%) effect on USP30. These results are all the more striking given that the concentration of hD1 inhibitor used was present at 5-10× molar excess and at a concentration that was 50-fold higher than the apparent Ki for USP22. Together, these results showed that hD1 is a specific inhibitor of the human DUB module.

### Example 5

### Cyclic Peptide Inhibitor Increases H2B Ubiquitination in Cells

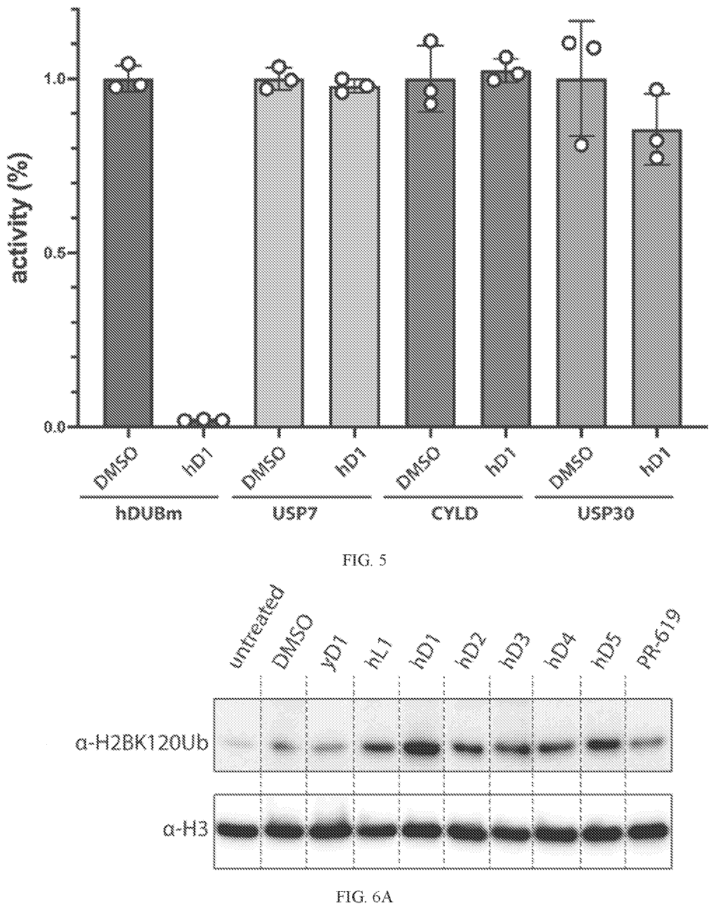

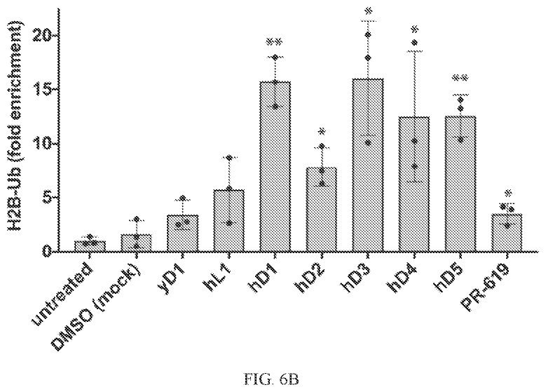

Each cyclic peptide was introduced as a supplement to the growth medium of HEK293T cells, the histones were extracted from each culture after two hours of exposure, and the levels of H2B-Ub was quantified by immunoblot. Levels of H2B-Ub were compared to control cells that were treated with DMSO, the yD1 cyclic peptide that has no effect on USP22 activity, or the broad-spectrum cell permeable DUB inhibitor. PR-619. The relative abundance of histone H3 was used to control for differences in nuclear extraction. As shown in FIG. 6A, treatment with five of the six inhibitors significantly increased H2B-Ub levels as compared to the controls. The most dramatic results were observed for hD1 and hD3, which increased H2B-Ub levels in excess of 15-fold, followed by hD4 and hD5, which increased H2B-Ub levels by about 12-fold. The hD2 and hL1 peptides resulted in a minor increase of H2B-Ub on the scale seen with the yD1 control peptide and the DUB inhibitor, PR-619. The robust effect of hD4 in the cell-based assay was interesting given that this was the least effective inhibitor in the Ub-AMC cleavage assay, with an apparent Ki of 804 μM. Overall, the accumulation of H2B-Ub indicated that the inhibitors are able to enter cells and inhibit DUB module activity.

Specifically targeting one or a subset of the ˜90 human DUBs with inhibitors is difficult because of the high degree of structural similarity within each superfamily. Identifying specific inhibitors of the USP family is particularly challenging because there are 55 USP DUBs, which share a structurally conserved catalytic domain and active site. Using in vitro selection from a library of cyclic peptides, inhibitors of USP22, a subunit of the SAGA deubiquitinating module whose overexpression is part of an 11-gene “death by cancer” signature, were identified.

The most potent of the inhibitors, hD1, inhibited USP22 in vitro with a Ki of 20.6 nM (FIG. 2C). Importantly, hD1 is highly specific and has little to no effect on the activity of several other human DUBs (FIG. 5). The ability of the peptide inhibitors to increase ubiquitination in cells (FIG. 6) indicated that the inhibitor is able to cross the outer cell membrane and enter the nucleus.

Interestingly, the effect in cells was not strictly correlated with the relative potency of each inhibitor in vitro; the weakest inhibitor of the peptides tested, hD4, had a similar effect on enriching H2B-Ub in cells to the most potent inhibitor, hD1, suggestive of each peptide entering cells with a different efficiency. Nevertheless, it is remarkable that 5 μM of the macrocyclic peptides disclosed herein, such as hD1, clearly enriched H2B-Ub in cells, suggesting that the inhibitor is active in cells and is membrane permeable at a level comparable to polycationic peptides, so-called cell-penetrating peptides.

### Example 6

### Characterization of Inhibition

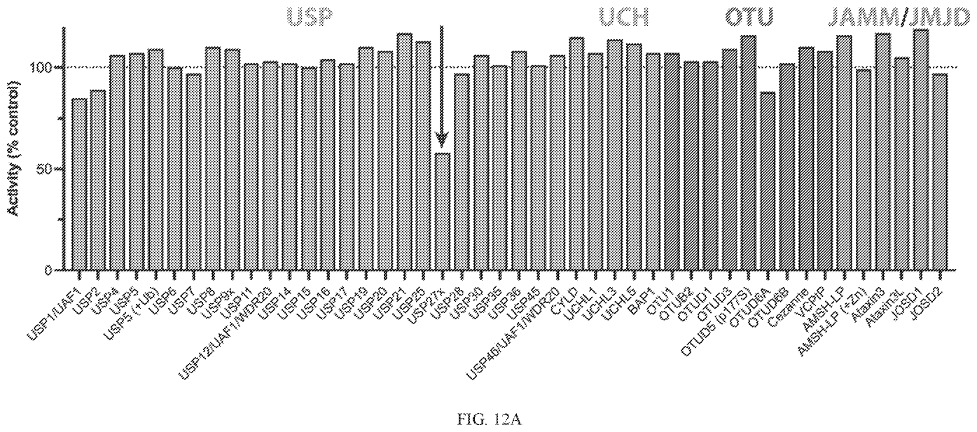

DUBm activity as a function of substrate concentration was assayed in the presence of several different concentrations of hD1 and then fit the data to competitive, non-competitive, and uncompetitive models of inhibition (FIG. 11A). The best fit was obtained with a non-competitive model, suggesting that hD1 does not compete for ubiquitin binding, but instead exerts its effect on the catalytic step of the reaction. Data from these experiments indicate that the Ki of hD1 is ˜180 nM, while Km was calculated to be 35 μM and kcat was 2.8 s⁻¹, matching previous experiments.

A rationale for using small, cyclized peptides as inhibitors is that cyclization constrains the conformation of the peptide backbone, thus reducing the unfavorable entropic penalty upon formation of a complex with the target enzyme. The ability of a linear hD1 peptide was tested to inhibit USP22. As shown in FIG. 11B, linear hD1 inhibits USP22 at a ˜4-fold higher concentration, indicating the constraining the peptide does not play a major role in the effectiveness of this inhibitor. To verify that the peptide sequence itself is important, rather than overall amino acid composition, we tested a peptide of the same length but with a scrambled amino acid sequence and showed that it failed to inhibit DUB activity, even at very high peptide concentrations (FIG. 11B).

### Example 7

### Selectivity for USP22

The human genome encodes about 80 active DUBs, 55 of which belong to the same USP structural family as USP22, thus presenting a challenge to the development of specific DUB inhibitors. To probe the relative specificity of hD1, a panel of 44 human DUBs was screened for inhibition, including 27 USP DUBs (DUBprofilerTM; Ubiquigent). The activity of each DUB was tested in the presence of a single concentration of inhibitor and compared with enzyme activity in the absence of compound. 1 μM was chosen as the concentration of hD1 to test, as the USP22/DUB module has no detectable activity under these conditions (FIG. 2C). As shown in FIG. 12A, hD1 is strikingly specific: it did not significantly affect the activity of any of the enzymes tested except for USP27x, whose activity was reduced by ˜50%. Interestingly, USP27x has been reported to deubiquitinate histone H2B-K120 in vivo and in vitro as part of a complex with ATXN7L3 and ENY2, two of the three adapter proteins that are part of the USP22 DUB module. In this screen, however, USP27x alone was tested in the absence of other subunits.

The relative specificity of hD1 was compared on USP22, USP27x, and USP51 in the context of their native complexes. Heterotrimeric complexes containing USP27x and USP51 were purified, respectively, in complex with ENY2 and ATXN7L3. The activity of these complexes on Ub-AMC was compared to that of the human DUBm, which contains USP22, ENY2, ATXN7L3, and a fourth subunit, ATXN7, that anchors the DUBm to the rest of the SAGA complex. As shown in FIG. 12B, hD1 inhibits the USP27x complex at >25-fold higher inhibitor concentrations and modestly inhibits the USP51 complex only at >2500-fold higher concentration than that needed to inhibit the human DUB module. The high specificity of hD1 for USP22 as compared to USP27x and USP51 is all the more notable given that all three complexes deubiquitinate nucleosomal histone H2B-K120Ub. In addition, the finding that hD1 inhibits USP27x alone as well as the USP27x/ENY2/ATXN7L3 complex suggest that hD1 inhibits DUB activity by binding directly to the catalytic domain, rather than to the ENY2 and ATXN7L3 subunits.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A cyclic peptide, or a pharmaceutically acceptable salt or solvate thereof, comprising an amino acid sequence with at least 50% similarity to any of SEQ ID NOs: 1-6.

2. The cyclic peptide of claim 1, comprising an amino acid sequence with at least 70% similarity to any of SEQ ID NOs: 1-6.

3. The cyclic peptide of claim 1 or 2, comprising an amino acid sequence of any of SEQ ID NOs: 1-6.

4. The cyclic peptide of any of claims 1-3, wherein a covalent linkage is between amino acids at N-terminus and C-terminus.

5. The cyclic peptide of claim 4, wherein the covalent linkage is a thioether bond.

6. The cyclic peptide of any of claim 1-5, wherein the cyclic peptide is selected from:

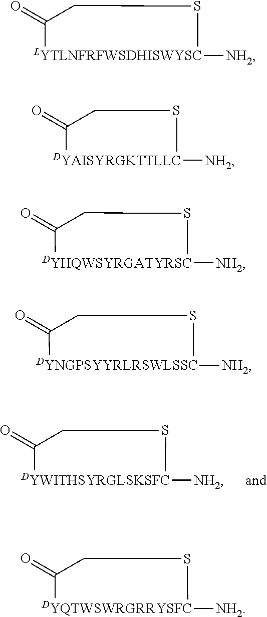

7. A composition comprising the cyclic peptide of any of claims 1-6.

8. The composition of claim 7, further comprising a carrier.

9. The composition of claim 7 or 8, further comprising a buffer.

10. A method for modulating histone H2B ubiquitination, the method comprising contacting a deubiquitinating enzyme with an effective amount of the peptide of any of claims 1-6, or the composition of any of claims 7-9.

11. The method of claim 10, wherein modulating histone H2B ubiquitination causes an increase in histone H2B ubiquitination.

12. The method of claim 10 or 11, wherein the deubiquitinating enzyme comprises Ubiquitin-Specific Protease 22 (USP22).

13. The method of any of claims 10-12, wherein the deubiquitinating enzyme comprises a member of a deubiquitinating protein complex.

14. The method of claim 13, wherein the deubiquitinating protein complex is SAGA (Spt-Ada-Gcn5-acetyltransferase) deubiquitinating module (DUBm).

15. The method of claim 13 or 14, wherein the deubiquitinating protein complex comprises USP 22, ataxin 7 (ATXN7), ataxin-7-like protein 3 (ATXN7L3), and transcription and mRNA export factor ENY2.

16. The method of any of claims 10-15, wherein the deubiquitinating enzyme is within a biological cell.

17. The method of any of claims 10-16 wherein the biological cell is ex vivo or in an organism.

18. A method of treating a disease or disorder comprising administering to a subject in need thereof an effective amount of the peptide of any of claims 1-6 or the composition of any of claims 7-9.

19. The method of claim 18, wherein the disease or disorder comprises cancer.

20. The method of claim 18 or 19, wherein the disease or disorder comprises brain cancer, breast cancer, stomach cancer, liver cancer, lung cancer, and colorectal cancer, or any combination thereof.
